(19) United States
(12) Patent Application Publication
(10) Pub. No.: US 2008/0177298 A1
(43) Pub. Date: Jul. 24, 2008

(54) Tensioner Tool and Method for Implanting an Interspinous Process Implant Including a Binder

(75) Inventors: James F. Zucherman, San Francisco, CA (US); Ken Y. Hsu, San Francisco, CA (US); Charles J. Winslow, Walnut Creek, CA (US); Scott A. Yerby, Montara, CA (US); Steven T. Mitchell, Pleasant Hill, CA (US); John J. Flynn, Walnut Creek, CA (US); John A. Markwart, Castro Valley, CA (US)

Correspondence Address: COATS & BENNETT/MEDTRONICS, 1400 CRESCENT GREEN, SUITE 300, CARY, NC, 27518, US

(73) Assignee: ST. FRANCIS MEDICAL TECHNOLOGIES, INC., Sunnyvale, CA (US)

(21) Appl. No.: 11/874,862

(22) Filed: Oct. 18, 2007

Related U.S. Application Data
(60) Provisional application No. 60/853,957, filed on Oct. 24, 2006.

(51) Int. Cl. A61B 17/00

(52) U.S. Cl. 606/207; 606/205

(57) ABSTRACT

Systems in accordance with embodiments of the present invention can include an implant comprising a spacer for defining a minimum space between adjacent spinous processes, a distraction guide for piercing and distracting an interspinous ligament during implantation, a binder for limiting or preventing flexion motion of the targeted motion segment, and a tensioner tool for applying tension to the binder. In one embodiment, the tensioner engages the implant and draws the binder through the capture device and applies tension to the binder. In one embodiment, the tensioner tool has a tension gauge with which a physician may measure the tension applied to the binder. The binder may be grasped by the capture device when the desired tension is achieved.

### CROSS-REFERENCES TO RELATED APPLICATIONS

This U.S. patent application claims the benefit under 35 U.S.C. §109(e) of U.S. Provisional Patent Application No. 60/853,957, as filed on Oct. 24, 2006, the full disclosure of which is incorporated herein by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

As the present society ages, it is anticipated that there will be an increase in adverse spinal conditions which are characteristic of people as they age. Certain biochemical changes can occur with aging which affect tissue found throughout the body. In the spine, the structure of the intervertebral disks can be compromised, in part as the structure of the annulus fibrosus of the intervertebral disk weakens due to degenerative effects. Spondylosis (also referred to as spinal osteoarthritis) is one example of a degenerative disorder that can cause loss of normal spinal structure and function. The degenerative process can impact the cervical, thoracic, and/or lumbar regions of the spine, affecting the intervertebral disks and the facet joints. Pain associated with degenerative disorders is often triggered by one or both of forward flexion and hyperextension. Spondylosis in the thoracic region of the spine can cause disk pain during flexion and facet pain during hyperextension. Spondylosis can affect the lumbar region of the spine, which carries most of the body's weight, and movement can stimulate pain fibers in the annulus fibrosus and facet joints.

Over time, loss of disk height can result in a degenerative cascade with deterioration of all components of the motion segment resulting in segment instability and ultimately in spinal stenosis (including, but not limited to, central canal and lateral stenosis). Spinal stenosis results in a reduction in foraminal area (i.e., the available space for the passage of nerves and blood vessels) which compresses the nerve roots and causes radicular pain. Another symptom of spinal stenosis is myelopathy. Extension and ipsilateral rotation further reduces the foraminal area and contributes to pain, nerve root compression and neural injury. During the process of deterioration, disks can become herniated and/or become internally torn and chronically painful. When symptoms seem to emanate from anterior (disk) and posterior (facets and foramen) structures, patients cannot tolerate positions of extension or flexion.

A common procedure for handling pain associated with degenerative spinal disk disease is the use of devices for fusing together two or more adjacent vertebral bodies. The procedure is known by a number of terms, one of which is interbody fusion. Interbody fusion can be accomplished through the use of a number of devices and methods known in the art. These include screw arrangements, solid bone implant methodologies, and fusion devices which include a cage or other mechanism which is packed with bone and/or bone growth inducing substances. All of the above are implanted between adjacent vertebral bodies in order to fuse the vertebral bodies together, alleviating associated pain.

Depending on the degree of slip and other factors, a physician may fuse the vertebra “as is,” or fuse the vertebrae and also use a supplemental device. Supplemental devices are often associated with primary fusion devices and methods, and assist in the fusion process. Supplemental devices assist during the several month period when bone from the adjacent vertebral bodies is growing together through the primary fusion device in order to fuse the adjacent vertebral bodies. During this period it is advantageous to have the vertebral bodies held immobile with respect to each other so that sufficient bone growth can be established. Supplemental devices can include hook and rod arrangements, screw arrangements, and a number of other devices which include straps, wires, and bands, all of which are used to immobilize one portion of the spine relative to another. Supplemental devices have the disadvantage that they generally require extensive surgical procedures in addition to the extensive procedure surrounding the primary fusion implant. Such extensive surgical procedures include additional risks, including risk of causing damage to the spinal nerves during implantation. Spinal fusion can include highly invasive surgery requiring use of a general anesthetic, which includes additional risks. Risks further include the possibility of infection, and extensive trauma and damage to the bone of the vertebrae caused either by anchoring of the primary fusion device or the supplemental device. Finally, spinal fusion can result in an absolute loss of relative movement between vertebral bodies.

It would be advantageous if a device and procedure for limiting flexion and extension of adjacent vertebral bodies were as simple and easy to perform as possible, and would preferably (though not necessarily) leave intact all bone, ligament, and other tissue which comprise and surround the spine. Accordingly, it would be desirable to have surgical devices and implantation procedures for limiting flexion and extension of adjacent vertebral bodies that preserve the physiology of the spine.

It would also be desirable to have procedures and implants which are minimally-invasive and which can supplement or substitute for primary fusion devices and methods, or other spine fixation devices and methods.

It would be further desirable to have minimally-invasive surgical implantation methods for spine implants.

It would be still further desirable to have tools to facilitate minimally-invasive surgical implantation while minimizing further trauma to the spine, and obviating the need for invasive methods of surgical implantation.

### BRIEF DESCRIPTION OF THE DRAWINGS

The features of the invention, its nature and various advantages will be apparent from the accompanying drawings and the following detailed description of the preferred embodiments, in which:

FIG. 1A shows an exploded perspective view of an interspinous implant illustrating components that interact with a tensioner in accordance with one embodiment of the invention;

FIG. 1B shows a perspective view of the interspinous implant of FIG. 1A after assembly illustrating a binder that interacts with a tensioner in accordance with one embodiment of the invention;

FIG. 1C shows a sectional view of a capture device in accordance with one embodiment of the present invention;

FIG. 1D shows a lateral view of the interspinous implant and binder of FIGS. 1A and 1B in position between adjacent spinous processes;

FIG. 2A shows a top view of a tensioner in accordance with one embodiment of the invention;

FIG. 2B shows a side view of the jaws of the tensioner of FIG. 2A;

FIG. 2C shows a perspective view of the tensioner of FIG. 2A illustrating the fork and jaws;

FIG. 2D shows a sectional view of the fork of the tensioner engaging an implant and binder in accordance with one embodiment of the invention;

FIG. 3A shows a posterior partial sectional view of the interspinous implant of FIGS. 1A-D in position between adjacent spinous processes illustrating the location of the fork and jaws of the tensioner of FIGS. 2A-D relative to the implant and binder of FIGS. 1A-D;

FIG. 3B shows an anterior partial sectional view of the tensioner of FIGS. 2A-D illustrating interaction between the tensioner of FIGS. 2A-D and the implant and binder of FIGS. 1A-D; and

FIG. 4 is a flow chart diagram of a method of positioning the implant of FIGS. 1A-D between adjacent spinous processes and tensioning the band using the tensioner of FIGS. 2A-D.

### DETAILED DESCRIPTION OF THE INVENTION

In view of the foregoing background of the invention, it is an object of this invention to provide a device and implantation procedure for limiting flexion and extension of adjacent vertebral bodies that preserve the physiology of the spine.

It is also an object of this invention to provide procedures and implants which are minimally-invasive and which can supplement or substitute for primary fusion devices and methods, or other spine fixation devices and methods.

It is a further object of this invention to provide minimally-invasive surgical implantation methods for spine implants.

It is a still further object of this invention to provide tools to facilitate minimally-invasive surgical implantation while minimizing further trauma to the spine, and obviating the need for invasive methods of surgical implantation.

In accordance with the objects and background of the invention, in one embodiment, the present invention provides a system including a spacer for defining a minimum space between adjacent spinous processes, a distraction guide for piercing and distracting an interspinous ligament during implantation, a binder for limiting or preventing flexion motion of the targeted motion segment and a tensioner for precisely tensioning the binder. The binder can be secured to a brace associated with the implant by a capture device. In one embodiment, a tensioner provides for a measured application of tension to the binder prior to securing the binder with the capture device. In a particular embodiment the tensioner has jaws designed to interact with the implant to tension the binder and a tension gauge to measure the amount of tension applied to the binder.

Other implants, methods and surgical instruments within the spirit and scope of the invention can be used to relieve pain associated with the spine and/or increase the volume of the spinal canal. Additional objects, advantages, and embodiments of the invention are set forth in part in the description which follows, and in part, will be obvious from this description, or may be learned from the practice of the invention. The following description is of the best modes presently contemplated for practicing various embodiments of the present invention. The description is not to be taken in a limiting sense but is made merely for the purpose of describing the general principles of the invention. The scope of the invention should be ascertained with reference to the claims. In the description of the invention that follows, like numerals or reference designators will be used to refer to like parts or elements throughout. In addition, the left-most digit of a reference number identifies the drawing in which the reference number first appears.

### Interspinous Implant and Binder

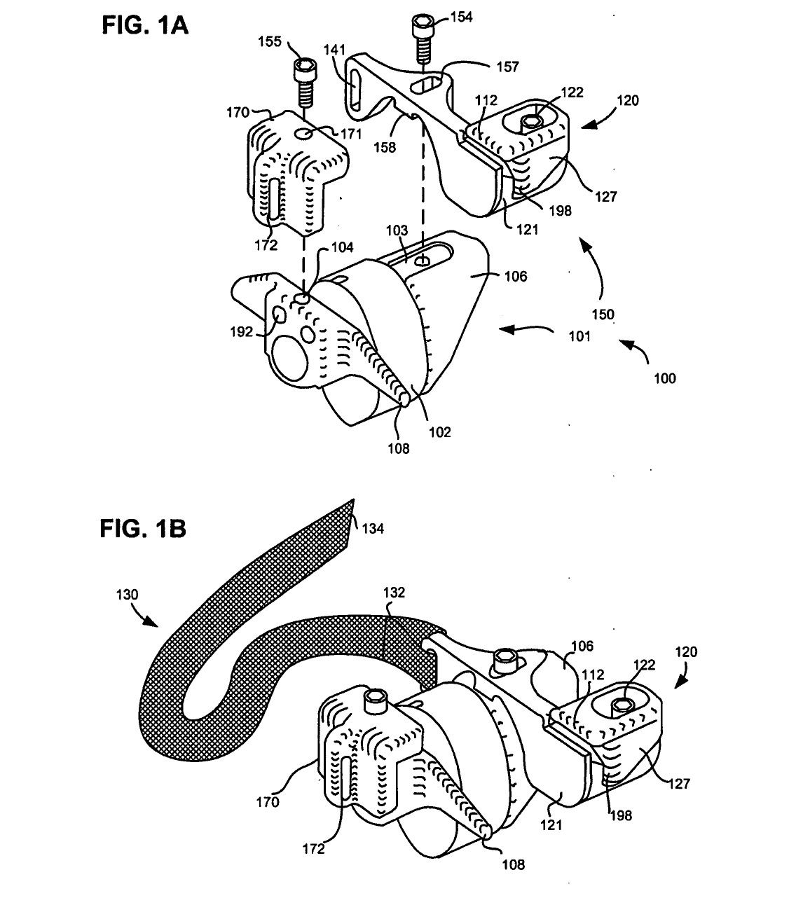

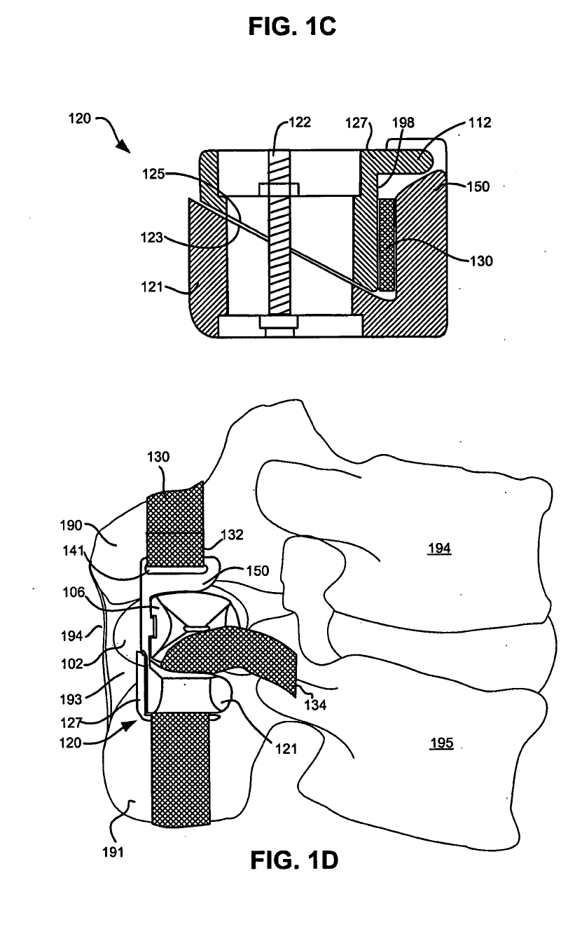

FIGS. 1A-D are views of an embodiment of an implant 100 in accordance with the present invention. In such an embodiment, the implant 100 can include a main body 101. The main body 101 (also referred to herein as a first unit) includes a spacer 102, a first wing 108, a distraction guide 106 and an alignment track 103. The main body 101 is inserted between adjacent spinous processes. Preferably, the main body 101 remains (where desired) in place without attachment to the bone or ligaments.

The alignment track 103 includes a threaded hole for receiving a fastener. The alignment track 103 need not include a threaded hole, but rather alternatively can include some other mechanism for fixedly connecting an additional piece (such as a second wing for limiting or blocking movement of an implant along the longitudinal axis). For example, in an alternative embodiment, the alignment track 103 can include a flange so that the second wing 150 can be slidably received.

As further shown in FIGS. 1A and 1B, the implant 100 includes a second wing 150 removably connectable with the implant 100. The second wing 150 includes an alignment tab 158 adapted to be received in the alignment track 103 of the main body 101. The alignment tab 158 optionally includes a slot for receiving the fastener so that the alignment tab 158 is disposed between the fastener and the alignment track 103. In alternative embodiments, the alignment tab 158 need not include a slot but rather can include some other mechanism for mating with the main body 101.

The second wing 150 can include a first end having a slot (or eyelet) 141 through which the proximal end (also referred to herein as an anchored end) 132 of a binder 130 can be threaded and subsequently sutured, knotted or otherwise bound, or alternatively looped through the slot 141 and secured to itself (e.g., using a clasp) so that the proximal end 132 of the binder 130 cannot be withdrawn through the slot 141. One of ordinary skill in the art can appreciate the myriad different ways in which the proximal end 132 of the binder 130 can be associated with the second wing 150 so that tension can be applied to the binder 130. The binder 130 can be disposed around adjacent spinous processes and a portion of the length of the binder 130 (the length of the binder being that portion of the binder extending from the proximal end of the binder) can be secured to the second wing 150 by a capture device 120 associated with the second wing 150.

The binder 130 can comprise a strap, ribbon, tether, cord, or some other flexible (or semi-flexible), and preferably threadable structure. The binder 130 can be made from a biocompatible material. In an embodiment, the binder 130 can be made from a braided polyester suture material. Braided polyester suture materials are non-absorbable, have high tensile strength, low tissue reactivity and improved handling. In other embodiments, the binder 130 can be made from stainless steel (i.e., surgical steel), which can be braided into a tether or woven into a strap, for example. In still other embodiments, the binder 130 can be made from some other material (or combination of materials) having similar properties.

FIG. 1C shows a sectional view of a capture device 120 in accordance with one embodiment of the present invention. The capture device 120 of FIGS. 1A-C is arranged at a second end of the second wing 150 opposite the slot 141. As illustrated in FIG. 1C, the capture device 120 can comprise, for example, two pieces slidably associated with one another by an adjustable fastener 122. A fixed piece 121 of the capture device extends from the second wing 150. The fixed piece 121 includes a beveled surface 123 that functions as a ramp. A slidable piece 127 of the capture device is slidably associated with the fixed piece 121, for example, via the adjustable fastener 122. Slidable piece 127 likewise includes a beveled surface 125 positioned in opposition to the beveled surface of the fixed piece 121. As the adjustable fastener 122 is tightened, slidable piece 127 slides along the beveled surface of the fixed piece 121, and the distance between the capture surface 198 of the slidable piece 127 and the second wing 150 decreases thereby securing the binder 130 between the two surfaces. As described above, the slidable piece 127 can optionally further include a guide 112 extending from the slidable piece 127 so that the guide 112 overlaps a portion of the second wing 150. The guide 112 can extend, for example, a distance roughly similar to the maximum distance between the capture surface 198 and the second wing 150, and can help ensure that the binder 130 is arranged between the capture surface 198 and the second wing 150.

In other embodiments, the capture device of FIGS. 1A-D can include some other shape, configuration, and mechanism and still fall within the contemplated scope of the invention. For example, in other embodiments, a flange can extend from the second wing 150, from which a rotatable cam or a spring-loaded cam extends so that the binder 130 can be captured between the second wing 150 and the cam. In still further embodiments in accordance with the present invention, some other mechanism can be employed as a capture device associated with the second wing 150 for securing the length of the binder 130. One of ordinary skill in the art will appreciate the myriad different mechanisms for securing the binder 130 to the second wing 150.

A physician can position the binder 130 so that the binder 130 is disposed between adjacent spinous processes, threading the binder 130 between the slidable piece 127 and the second wing 150. The physician can then adjust the fastener 122 so that the distance between the capture surface 198 and the second wing 150 decreases, thereby pinching the binder 130 between the capture surface 198 and the second wing 150 and defining a secure end of the binder 130. In some embodiments, one or both of the capture surface 198 and the second wing 150 can include texture so that the binder 130 is further prevented from sliding when the binder 130 is placed under increasing tension (e.g., during flexion).

The implant 100 can further include a binder aligner 170 selectably connectable with the first wing 108 of the main body 101. The binder aligner 170 can be connected with the first wing 108 by fastening the binder aligner 170 to a locking pin hole 104 of the first wing 108. In such embodiments where a fastener 155 is used to connect the binder aligner 170 with the first wing 108 through a hole 171 in the binder aligner 170, it is desirable that the locking pin hole 104 be threaded, or otherwise adapted to receive the fastener 155. The locking pin hole 104 can thus be adapted to function as a hole to slidably (and temporarily) receive a locking pin of an insertion tool (not shown), thereby facilitating insertion and positioning of the main body 101, and can also be adapted to function to fixedly receive a fastener 155 for positioning the binder aligner 170. The binder aligner 170 can optionally include corresponding to alignment holes 192 of the main body 101 to further secure the binder aligner 170 to the main body 101 and limit undesired movement of the binder aligner 170 relative to the main body 101.

The binder aligner 170 includes a guide 172 extending from the binder aligner 170 to limit or block shifting of the binder 130 in a posterior-anterior direction. The guide 172 can include a loop, as shown in FIG. 1A, or alternatively some other structure, closed or unclosed, for limiting or blocking shifting of the binder 130. Such a structure can prevent undesired relative movement between the binder 130 and the main body 101, and can additionally ease arrangement of the binder 130 during an implantation procedure, by helping to aid proper positioning of the binder 130.

Referring now to FIG. 1D in which the interspinous implant 100 is shown positioned between adjacent spinous process 190, 191. Note that spacer 102 is located between the two spinous processes 190, 191 and between the supra-spinous ligament 194 and the vertebrae 194, 195, and through the interspinous ligament 193. Binder 130 has been threaded around the spinous processes 190, 191 and through capture device 120. Note that the free end of binder 130 comes out the back of implant 100 between capture device 120 and distraction guide 106.

As will be readily apparent to one of skill in the art, implants in accordance with the present invention provide significant benefits to a physician by simplifying an implantation procedure and reducing procedure time, while providing an implant that can limit or block flexion and extension of the spine. A physician can position an implant between adjacent spinous processes and can position a binder 130 connected with the second wing around the spinous processes without requiring the physician to measure an appropriate length of the binder 130 prior to implantation. The capture device 120 allows the binder 130 to be secured to the second wing anywhere along a portion of the binder 130, the portion being between a distal end 134 of the binder 130 and the proximal end 132. The physician can secure the binder 130 to the second wing to achieve the desired range of movement (if any) of the spinous processes during flexion. Further details of interspinous implants, binders and capture devices which can be utilized as part of the present invention may be found in U.S. patent application, entitled “Interspinous Process Implant Including A Binder And Method Of Implantation,” filed Mar. 31, 2005, Ser. No. 11/095/440, and U.S. patent application, entitled “Interspinous Process Implant Including A Binder And Method Of Implantation,” filed Mar. 31, 2005, Ser. No. 11/095/680, both of which are incorporated herein by reference.

### Binder Tensioner

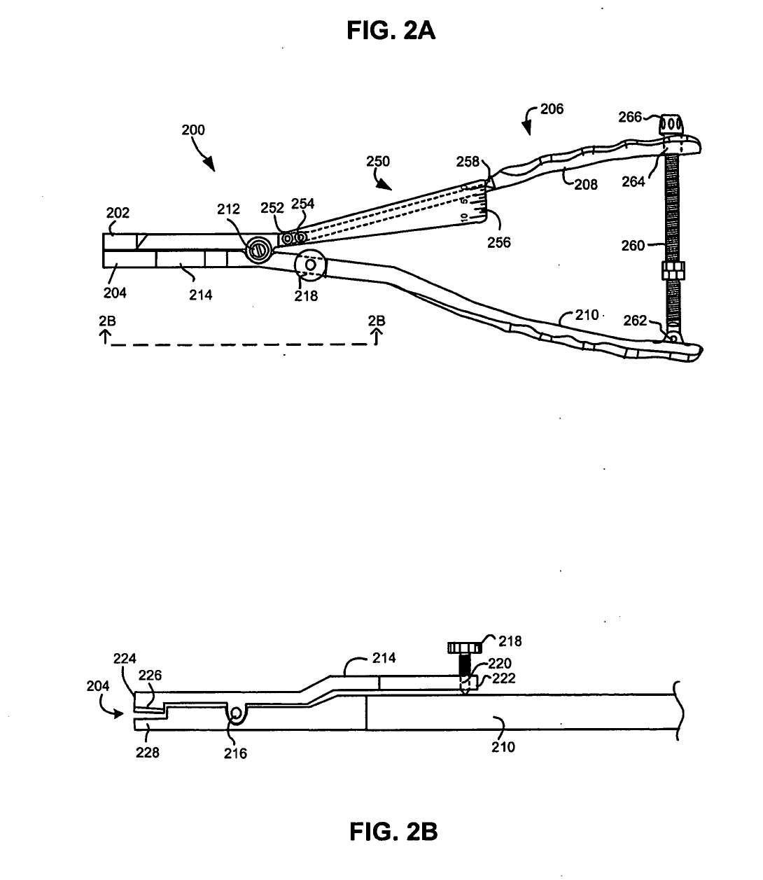

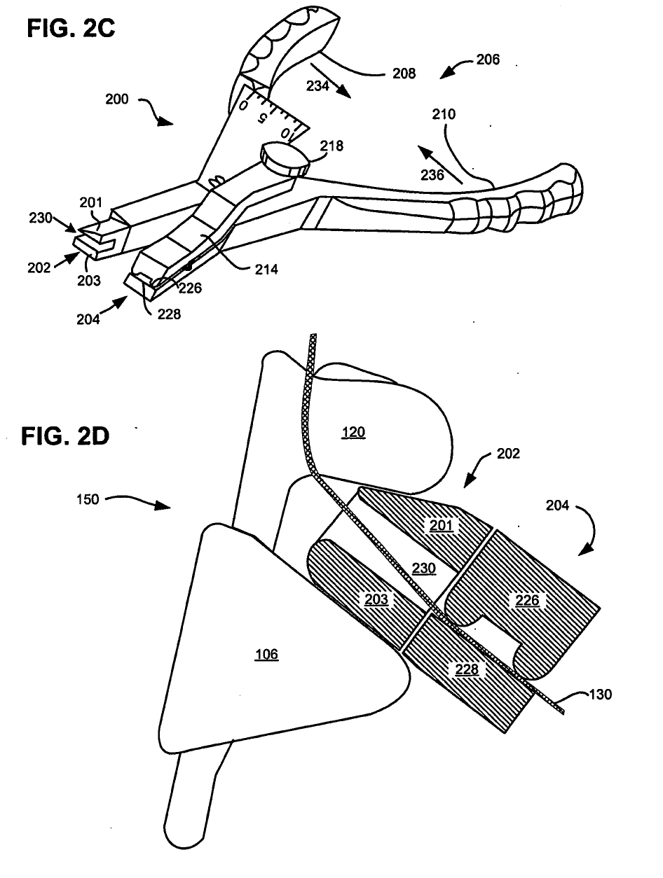

FIG. 2A is a plan view of an embodiment of a tensioner 200 in accordance with the present invention for use with embodiments of systems and methods of the present invention. The tensioner 200 includes a fork 202 and jaws 204 at the distal end. At the proximal end the tensioner 200 includes a handle (also referred to herein as a grip) 206. The handle 206 is defined by a first member 208 and a second member 210 pivotally connected by a fastener 212. As shown, the handle 206 is scalloped, or otherwise textured so that a physician can grip the handle 206 with less slippage than might otherwise occur. In other embodiments, the handle 206 need not be textured or scalloped, or can include some other feature for assisting proper handling. For example, in some embodiments, the handle 206 can include finger loops. In this embodiment, the distal end of the first member 208 is associated with fork 202 and the distal end of second member 210 is associated with jaws 204. The fork 202 and jaws 204 are positioned in opposition to one another.

In one embodiment of the present invention, the tensioner 200 can also include a tension gauge 250 connected with one of the first and second members 208, 210. As shown in FIG. 2A, the tension gauge 250 is attached to first member 208 by two screws 252, 254. At the other end of tension gauge 250 is a scale 256. First member 208 comprises a pointer 258 adjacent scale 256. Pointer 258 deflects relative to scale 256 depending upon the amount of flex in first member 208 between screw 254 and pointer 258. The scale 256 may be calibrated by methods known to those of skill in the art such that it identifies the tension applied between jaws 204 and fork 202 corresponding to the measured deflection at scale 256. First member 208 is designed such that pointer 258 deflects over scale 256 for a range of desirable tensions to be applied to a binder. For example, if the maximum tension to be applied is 200N, first member 208 may be designed such that full range deflection is achieved at 250N. Tensioner may alternatively include other devices known in the art to measure tension including, for example, an electronic strain gauge, piezoresistor or the like.

As further shown in FIG. 2A, in one embodiment the tensioner 200 can include a threaded rod 260 connected at a pivot point 262 to one of the first and second members 208, 210 and freely passing through a through-hole 264 in the other of the first and second members 208, 210. As the handle 206 is urged closed, the threaded rod 154 passes through the through-hole 264 and pivots to follow the arcing travel of the handle 206. A nut 266 can be associated with the threaded rod 260 at the free end of the threaded rod 260 such that when the nut 266 is advanced along the threaded rod 260 toward the pivot point 262, the nut 266 contacts the handle 206, fixing the handle 206 in position against tension applied between the fork 202 and jaws 204. Thus, a physician can urge the handle 206 closed to tension a binder and then fix the nut 266 against the handle 206 to lock the position of the handles and maintain the tension. The physician can then secure binder 130 with capture device 120 of implant 100 without having to maintain pressure on handle 206. For small incremental changes in tension, the nut 158 can be used as an actuator. The nut 158 can be twisted to urge the handle 206 closed or allow the handle 206 to open, depending on the direction of twist. In this way the nut 266 can permit careful and precise application of tension between fork 202 and jaws 204. The pitch of the threads on the threaded rod 260 can be sized such that a desired level of precision can be obtained when the nut 266 is advanced along the threaded rod 260. However, in other embodiments of tensioner 200 no threaded rod and nut is present or required. In still other embodiments, the tensioner 200 can include a pawl and ratchet mechanism in substitution for the threaded rod 260 and nut 266. Tensioners 200 in accordance with the present invention can include myriad different devices fixing a particular applied tension. Such tension locks are preferably designed so as not to preclude measurement of the tension in binder 130.

Referring now to FIG. 2B which shows a side view of the distal end of second member 210. Jaws 204 comprise in one embodiment, the distal end of second member 210 and a clamping member 214. Clamping member 214 and second member 210 are pivotally connected by a fastener 216. Clamping screw 218 passes through a threaded hole 220 in the proximal end of clamping member 214 and contacts the surface of second member 210 such that by turning clamping screw 218, proximal end 222 of clamping member 214 can be forced away from second member 210. At the same time the distal end 224 of clamping member 214 is forced towards second member 210 in the region of jaws 204.

Jaws 204 include jaw 226 and jaw 228 that oppose and abut one another when the distal end 224 of clamping member 214 is biased towards second member 210 by clamping screw 218. Jaw 228 is part of member 210 and is the stationary component of jaws 204. Jaw 226 is part of clamping member 214 and is moved relative to member 210 by operation of clamping screw 218. Jaws 204 are adapted thereby to securely grasp a binder between jaw 226 and jaw 228 and prevent slippage of the binder during application of tension. Jaw 226 and jaw 228 may also be provided with surface features such as ridges, protrusions, roughening and the like to prevent slippage of the binder 130.

FIG. 2C shows a perspective view of tensioner 200 with the distal end closest to the viewer. In this view it can be seen that fork 202 comprises a slot 230 in-line with meeting point of jaw 226 and jaw 228 of jaws 204. In addition, fork 202 tapers away from jaws 204. In this embodiment, fork 202 comprises two tines 201 and 203 with slot 230 passing between tine 201 and tine 203.

As can be seen in more detail in FIG. 2D, the shape of fork 202 is selected so that fork 202 aligns slot 230 with second wing 150 of implant 100. The surfaces of tine 201 and tine 203 that contact implant 100 comprise an engagement surface that engages second wing 150. FIG. 2D illustrates the path of binder 130 through second wing 150 and the tensioner 200. Note that binder 130 may pass freely from its exit point at the back of second wing 150, through slot 230 and into jaws 204. In this embodiment, tine 201 is tapered so as to engage capture device 120 at an angle which aligns slot 230 with the path of the binder 130. Tine 203 is also adapted to engage distraction guide 106 as tine 201 engages the capture device 120. The shape of fork 202 is thus, complementary to the geometry of the implant in the region where the fork engages the implant. The shapes of tines 201 and 203 enable fork 202 to engage the second wing 150 in a manner that allows for secure temporary engagement of the fork and implant. The engagement of implant 100 by fork 202 is stable both as to position and the angle. Application of tension to the binder increases the force between the engagement surface of tines 201, 203 and implant 100 but does not push the tensioner away from the stable position and angle of engagement. By controlling the angle and position of fork 202 and jaws 204 the tensioner minimizes the resistance to movement of the binder 130 and facilitates measurement of tension in the binder. In other embodiments, the jaws 204 and fork 202 can have some other shape that is complementary with the geometry of the particular implant with which it is used. One of ordinary skill in the art can appreciate the myriad different shapes with which the jaws 204 and fork 202 can be formed.

Referring again to FIG. 2C, in operation, the binder is threaded through slot 230 of fork 202 and between jaw 226 and jaw 228 of jaws 204 with the handle 206 in its fully open position. With the handle 206 in its fully open position fork 202 abuts jaws 204 (as shown in FIG. 2A). When binder 130 is in position, the clamping screw 218 is tightened causing jaw surface 226 to be forced towards jaw 228 to securely grasp the binder 130. The handle 206 may then be urged towards its closed position as shown by arrows 234, 236 in FIG. 2C pushing jaws 204 away from fork 202 and pulling the binder through slot 230.

A system in accordance with the present invention can comprise a main body, a second wing 150 including a capture device 120 as described above, a binder, optionally a binder aligner 170 and a tensioner 200 designed to engage the implant 100 and grasp the binder 130. Alternatively, the system can be used to attach a binder 130 to a main body 101 previously implanted in a patient, for example by removing an existing second wing 150 and replacing the original second wing with a second wing 150 including a capture device 120 as described above, a binder and optionally a binder aligner 170 to additionally limit flexion as well as extension. In this case, the main body is not replaced during the procedure and thus no additional main body is required to be provided. Such a system can provide flexibility to a physician by allowing the physician to configure or reconfigure an implant according to the needs of a patient. Further, such a system can reduce costs by reducing the variety of components that need be manufactured to accommodate different procedures and different treatment goals.

### Operation of the Binder Tensioner

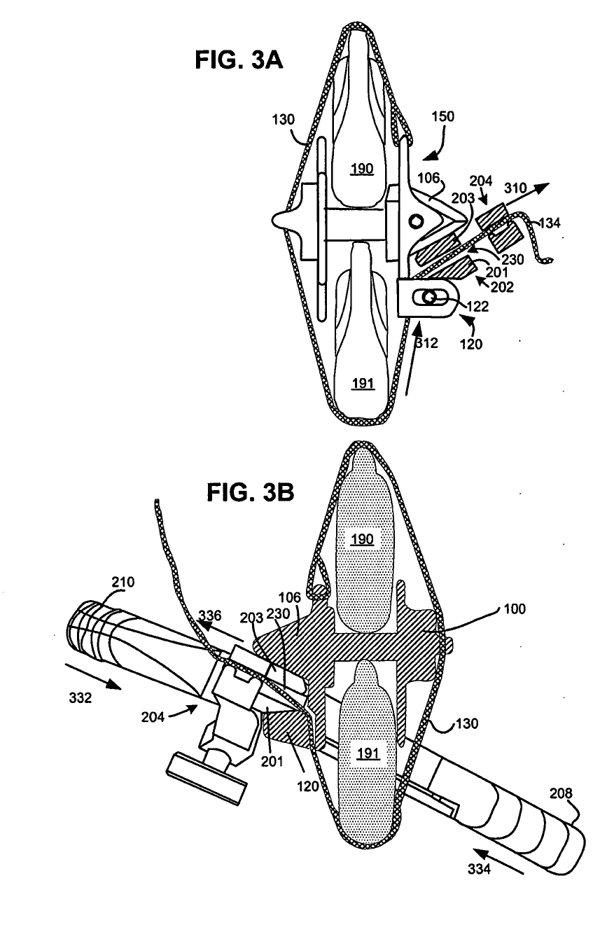

In one embodiment tensioner 200 is specifically adapted for operation in conjunction with an implant 100. FIG. 3A is a posterior view of an implant 100 and binder 130 in position between adjacent spinous processes 190, 191. As shown in FIG. 3A, the distal end 134 of binder 130 is threaded through capture device 120 and exits between capture device 120 and distraction guide 106. Fork 202 of tensioner 200 is designed to fit the shape of the space between capture device 120 and distraction guide 106 such that fork 202 can stably engage implant 100 while aligning slot 230 with the binder 130. It is desirable to align slot 230 with binder 130 so that there is as little possible resistance to the axial movement of the binder through the capture device 120 and slot 230 with the capture device 120 in the open position. The resistance should be minimized to facilitate tensioning of the binder 230. The resistance should also be minimized so that the tension measured at tension gauge 250 is a more accurate representation of the actual tension in the binder 130.

Referring again to FIG. 3A, a sectional view of fork 202 and jaws 204 is shown to illustrate the interaction of fork 202 and jaws 204 with implant 100. Fork 202 is complementary with the geometry of the implant in the region where it is desired that the tensioner stably engage the implant. As shown in FIG. 3A, the tine 201 of fork 202 is tapered so as to fit against capture device 120 while aligning slot 230 with binder 130. Tine 203 of fork 202 is adapted to fit against distraction guide 106. Jaws 204 are likewise aligned with slot 230 such that when handle 206 of tensioner 200 is urged closed, jaws 204 will move away from fork 202 in the direction of arrow 3 10. Binder 130 will thus be drawn through capture device 120 and slot 230 until it is tight around spinous processes 190, 191. When binder 130 is tight around spinous processes 190, 191, further application of closing pressure on handle 206 will apply tension to binder 130. Because fork 202 is complementary with the geometry of the implant, the tensioner does not move away from the correct engagement position and alignment when tension is applied to binder 130.

Referring to FIG. 3B in which an anterior view of tensioner 200 is shown relative to a dorsal section through adjacent spinous processes 190 and 191, implant 100, and binder 130. As can be observed fork 202 fits into the space between tissue distractor 106 and capture device 120 while aligning slot 230 with the path of binder 130 through capture device 120. As can further be seen, handle 206 of tensioner 200 is approximately perpendicular to the dorsal plane, thus fork 202 and jaws 204 can be properly positioned adjacent implant 100 through a small incision with handle 206 of tensioner 200 remaining outside of the patient's body as is desirable for minimally-invasive surgery. Referring again to FIG. 3B, closing of handle 206 by urging first member 208 towards second member 210 as shown by arrows 332, 334 forces jaws 204 away from fork 202 in the direction of arrow 336 thereby pulling binder 130 through slot 230 and capture device 120 and applying tension to binder 130.

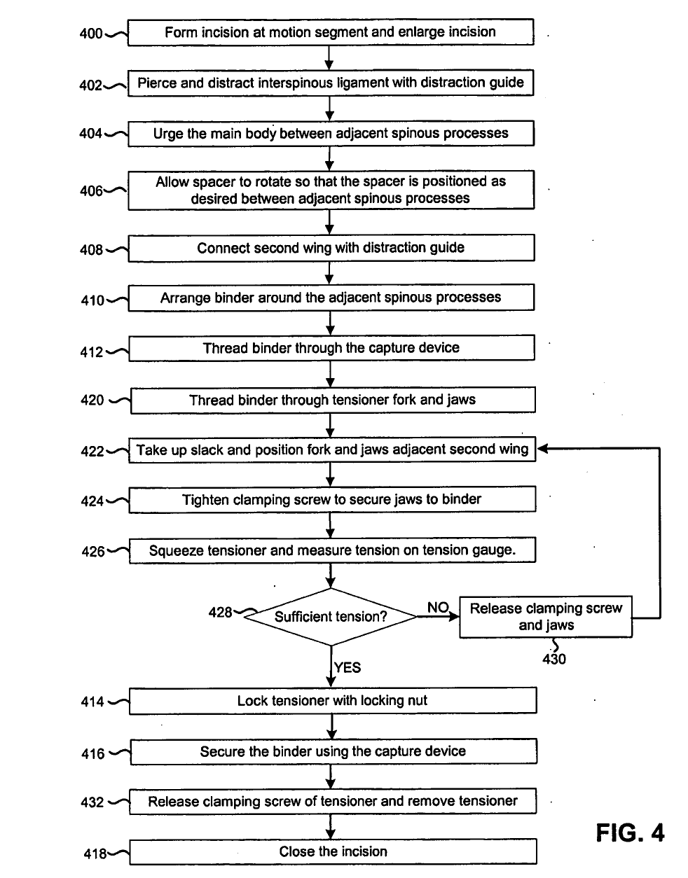

Referring now to FIG. 4 comprises a block diagram illustrating one embodiment of a method of surgically implanting an implant 100 utilizing a tensioner 200 as described above with respect to FIGS. 1A-D, 2A-D and 3A-B. The method can include forming an incision at the target motion segment, and enlarging the incision to access the target motion segment (Step 400). The interspinous ligament between targeted adjacent spinous processes can then be distracted by piercing or displacing the interspinous ligament with the distraction guide 106 (Step 402) and urging the implant 100 between the adjacent spinous processes (Step 404). As the interspinous ligament is displaced, the spacer 102 can be positioned between the spinous processes such that the spacer 102 can rotate to assume a preferred position between the spinous processes (Step 406). Once the implant 100 is positioned, the second wing 150 can be fixedly connected to the distraction guide 106 (Step 408). A binder 130 associated with the second wing 150 can then be threaded between interspinous ligaments of adjacent motion segments so that the targeted adjacent spinous processes are disposed within a loop formed by the binder 130 (Step 410). The physician can then thread the binder 130 between the capture surface 198 of the capture device 120 and the second wing 150 (Step 412). The physician can then thread the binder 130 through the slot 230 of fork 202 and between the grasping surfaces of jaws 204 (Step 420). With the handle 206 in the fully open position, the physician can then position the fork 202 of tensioner 200 against implant 100 in the space between capture device 120 and distraction guide 106 while pulling any slack in the binder through the jaws 204 and fork 202 (Step 422). The physician can then tighten the clamping screw 218 thereby clamping jaw 226 to jaw 228 of jaws 204 to binder 130 (Step 424). The physician can then urge the handle 206 closed (manually or with nut 266 if present) to tighten the binder while measuring the tension on the tension gauge 250 (Step 426). The physician can then evaluate whether enough tension has been achieved (Step 428). If sufficient tension has not been achieved, the physician may release clamping screw 218 and repeat the procedure starting with Step 422 by taking up more slack in binder 130. Once a desired tension of the binder 130 has been achieved as measured on tension gauge 250 (Step 428), if a threaded rod and nut 266 is provided, the physician may first adjust the position of nut 266 to maintain and/or incrementally adjust the tension. This will facilitate maintaining the proper tension while binder 130 is secured between the capture surface 198 and the second wing 150. The physician can then adjust the fastener 122 of the capture device 120 so that the binder 130 is secured between the capture surface 198 and the second wing 150 (Step 416). The physician may then release clamping screw 218 of the tensioner thereby allowing binder 130 to be removed from slot 230 and jaws 204. The physician may then remove the tensioner 200 from the patient (Step 432). The incision can subsequently be closed using standard surgical techniques (Step 418).

### Materials for Use in the Present Invention

In some embodiments, the implant and/or tensioner can be fabricated in whole or in part from medical grade metals such as titanium, stainless steel, cobalt chrome, and alloys thereof, or other suitable material having similar high strength and biocompatible properties. Additionally, the implant and/or tensioner can be at least partially fabricated from a shape memory metal, for example Nitinol, which is a combination of titanium and nickel. Thus, the tensioner 200 can be made sufficiently strong to support the force required to be applied during tensioning of binder 130. Such materials are typically radiopaque, and appear during x-ray imaging, and other types of imaging. Tensioner 200 may also be manufactured so as to be sterilizable and reusable.

Implants in accordance with the present invention, and/or portions thereof can also be fabricated from somewhat flexible and/or deflectable material. In these embodiments, the implant and/or portions thereof can be fabricated in whole or in part from medical grade biocompatible polymers, copolymers, blends, and composites of polymers. A copolymer is a polymer derived from more than one species of monomer. A polymer composite is a heterogeneous combination of two or more materials, wherein the constituents are not miscible, and therefore exhibit an interface between one another. A polymer blend is a macroscopically homogeneous mixture of two or more different species of polymer. The implant and/or portions thereof can be formed by extrusion, injection, compression molding and/or machining techniques.

Many polymers, copolymers, blends, and composites of polymers are radiolucent and do not appear during x-ray or other types of imaging. Implants comprising such materials can provide a physician with a less obstructed view of the spine under imaging, than with an implant comprising radiopaque materials entirely. However, the implant need not comprise any radiolucent materials.

One group of biocompatible polymers is the polyaryletherketone group which has several members including polyetheretherketone (PEEK), and polyetherketoneketone (PEKK). PEEK is proven as a durable material for implants, and meets the criterion of biocompatibility. Medical grade PEEK is available from Victrex Corporation of Lancashire, Great Britain under the product name PEEK-OPTIMA. Medical grade PEKK is available from Oxford Performance Materials under the name OXPEKK, and also from CoorsTek under the name BioPEKK. These medical grade materials are also available as reinforced polymer resins, such reinforced resins displaying even greater material strength. In an embodiment, the implant can be fabricated from PEEK 450G, which is an unfilled PEEK approved for medical implantation available from Victrex. Other sources of this material include Gharda located in Panoli, India. PEEK 450G has appropriate physical and mechanical properties and is suitable for carrying and spreading a physical load between the adjacent spinous processes. PEEK 450G has the following approximate properties:

| Property | Value |
|---|---|
| Density | 1.3 g/cc |
| Rockwell M | 99 |
| Rockwell R | 126 |
| Tensile Strength | 97 MPa |
| Modulus of Elasticity | 3.5 GPa |
| Flexural Modulus | 4.1 GPa |

It should be noted that the material selected can also be filled. Fillers can be added to a polymer, copolymer, polymer blend, or polymer composite to reinforce a polymeric material. Fillers are added to modify properties such as mechanical, optical, and thermal properties. For example, carbon fibers can be added to reinforce polymers mechanically to enhance strength for certain uses, such as for load bearing devices. In some embodiments, other grades of PEEK are available and contemplated for use in implants in accordance with the present invention, such as 30% glass filled or 30% carbon filled grades, provided such materials are cleared for use in implantable devices by the FDA, or other regulatory body. Glass filled PEEK reduces the expansion rate and increases the flexural modulus of PEEK relative to unfilled PEEK. The resulting product is known to be ideal for improved strength, stiffness, or stability. Carbon filled PEEK is known to have enhanced compressive strength and stiffness, and a lower expansion rate relative to unfilled PEEK. Carbon filled PEEK also offers wear resistance and load carrying capability.

As will be appreciated, other suitable similarly biocompatible thermoplastic or thermoplastic polycondensate materials that resist fatigue, have good memory, are flexible, and/or deflectable, have very low moisture absorption, and good wear and/or abrasion resistance, can be used without departing from the scope of the invention. As mentioned, the implant can be comprised of polyetherketoneketone (PEKK). Other material that can be used include polyetherketone (PEK), polyetherketoneetherketoneketone (PEKEKK), polyetheretherketone-ketone (PEEKK), and generally a polyaryletheretherketone. Further, other polyketones can be used as well as other thermoplastics. Reference to appropriate polymers that can be used in the implant can be made to the following documents, all of which are incorporated herein by reference. These documents include: PCT Publication WO 02/02158 A1, dated Jan. 10, 2002, entitled “Bio Compatible Polymeric Materials;” PCT Publication WO 02/00275 A1, dated Jan. 3, 2002, entitled “Bio Compatible Polymeric Materials;” and, PCT Publication WO 02/00270 A1, dated Jan. 3, 2002, entitled “Bio Compatible Polymeric Materials.” Other materials such as Bionate7, polycarbonate urethane, available from the Polymer Technology Group, Berkeley, Calif., may also be appropriate because of the good oxidative stability, biocompatibility, mechanical strength and abrasion resistance. Other thermoplastic materials and other high molecular weight polymers can be used. The spacer of the implant may also be made from natural or synthetic bone material.

The binder can be made from a biocompatible material. In one embodiment, the binder can be made from a braided polyester or Dacron suture material. Braided polyester and Dacron suture materials include, for example, ETHIBOND™, ETHIFLEX™, MERSILINE™ available from Ethicon, Inc., Cornelia, Ga., and are non-absorbable, having high tensile strength, low tissue reactivity and improved handling. In other embodiments, the binder can be made from stainless steel (i.e., surgical steel), which can be braided into a tether or woven into a strap, for example. In still other embodiments, the binder can be made from some other material (or combination of materials) having similar properties.

The foregoing description of the present invention has been presented for purposes of illustration and description. It is not intended to be exhaustive or to limit the invention to the precise forms disclosed. Many modifications and variations will be apparent to practitioners skilled in this art. The embodiments were chosen and described in order to best explain the principles of the invention and its practical application, thereby enabling others skilled in the art to understand the invention for various embodiments and with various modifications as are suited to the particular use contemplated. It is intended that the scope of the invention be defined by the following claims and their equivalents.

What is claimed is:

1. A surgical instrument comprising
an engagement surface adapted to be positioned against a surgical implant;
a jaws adapted to grasp a binder which passes through a portion of said surgical implant;
a handle that is operatively connected to the fork and the jaws;
said handle operable to move the jaws away from the engagement surface and thereby pull said binder through said portion of said surgical implant.

2. The surgical instrument of claim 1 comprising a fork having a first tine and a second tine separated by a slot wherein the fork comprises the engagement surface.

3. The surgical instrument of claim 2 wherein the engagement surface is adapted to engage the surgical implant such that the binder passes from the implant to the jaws through the slot.

4. The surgical instrument of claim 2 wherein the engagement surface is adapted to engage the surgical implant such that the angle and position of the slot relative to the implant is stable when tension is applied to the binder.

5. The surgical instrument of claim 1 wherein the engagement surface is complementary to a portion of the implant to be engaged by the engagement surface.

6. The surgical instrument of claim 1, including a gauge connected to the handle to assess a tension applied to the binder by the instrument.

7. The instrument of claim 1, including a locking mechanism that maintains the distance between the jaws and the engagement surface.

8. The instrument of claim 1, including an actuator which can operate the handle to urge the jaws away from the engagement surface.

9. The instrument of claim 1, wherein the jaws comprises a fixed jaw, a moving jaw and a clamping screw wherein the screw is operatively connected to the fixed jaw and the mobile jaw such that the screw is operable to urge the mobile jaw towards the fixed jaw thereby grasping the binder between the mobile jaw and the fixed jaw.

10. A system for immobilizing vertebral bodies by immobilizing respective spinous processes extending therefrom wherein the system comprises:
a surgical implant comprising a first wing, a spacer and a second wing wherein the second wing comprises an anchor and a capture device and wherein a binder is attached to the anchor;
a tool having an engagement surface adapted to engage the second wing, a jaws adapted to grasp the binder, and a handle operatively connected to the engagement surface and the jaws;
wherein operation of the handle of the tool draws the binder through the capture device.

11. The system of claim 10 wherein the tool comprises a fork having a first tine and a second tine separated by a slot wherein the fork comprises the engagement surface.

12. The system of claim 11 wherein the engagement surface is adapted to engage the surgical implant such that the binder passes from the implant to the jaws through the slot.

13. The system of claim 12 wherein the engagement surface is adapted to engage the surgical implant such that the angle and position of the slot relative to the implant is stable when tension is applied to the binder.

14. The system of claim 12 wherein the engagement surface is complementary to a portion of the implant to be engaged by the engagement surface.

15. The system of claim 10, wherein the tool includes a gauge connected to the handle to assess a tension applied to the binder by the instrument.

16. The system of claim 10, wherein the tool includes a locking mechanism that maintains the distance between the jaws and the engagement surface.

17. The system of claim 10, wherein the tool includes an actuator which can operate the handle to urge the jaws away from the engagement surface.

18. The system of claim 10, wherein the jaws comprises a fixed jaw, a moving jaw and a clamping screw wherein the screw is operatively connected to the fixed jaw and the mobile jaw such that the screw is operable to urge the mobile jaw towards the fixed jaw thereby grasping the binder between the mobile jaw and the fixed jaw.

19. A method of installing an implant, said implant comprising a binder wherein the method comprises the steps of:
(a) inserting the implant between body elements;
(b) positioning the binder around said body elements;
(c) engaging a body of the binder with a capture device of the implant;
(d) engaging the implant with a first portion of a tool;
(e) engaging the binder with a second portion of the tool;
(f) operating a handle of the tool to urge the first portion of the tool away from the second portion of the tool and thereby draw the binder through the capture device.

20. The method of claim 19, further comprising:
(g) reading a gauge of the tool to assess a tension of the binder.

21. The method of claim 20 further comprising:
(h) comparing the tension of the binder with a desired tension of the binder.

22. The method of claim 21, further comprising:
(j) adjusting the capture device to secure the body of the binder to the capture device.

23. The method of claim 22, further comprising:
(g1) adjusting a lock on the instrument to maintain the position of the first portion of the tool away from the second portion of the tool.

24. The method of claim 19, wherein the second portion of the tool comprises a fixed jaw, a moving jaw and a clamping screw wherein the screw is operatively connected to the fixed jaw and the mobile jaw; and wherein step e) comprises:
(e) operating the screw to urge the mobile jaw towards the fixed jaw and engage the binder by grasping the binder between the mobile jaw and the fixed jaw.
